(19) United States
(12) Patent Application Publication
(10) Pub. No.: US 2024/0366708 A1
(43) Pub. Date: Nov. 7, 2024

(54) TCM LUNG TEA METHOD AND COMPOSITION

(71) Applicant: Maishen Inc., Santa Clara, CA (US)

(72) Inventors: Dalong Zhang, Santa Clara, CA (US); Xinghe Chen, Santa Clara, CA (US); Eva Chen, Santa Clara, CA (US)

(73) Assignee: Maishen Inc., Santa Clara, CA (US)

(21) Appl. No.: 18/745,883

(22) Filed: Jun. 17, 2024

(51) Int. Cl. A61K 36/78; A61K 33/04; A61K 35/02; A61K 36/28; A61K 36/287; A61K 36/355; A61K 36/484; A61K 36/65; A61K 45/06

(52) U.S. Cl. CPC A61K 36/78; A61K 33/04; A61K 35/02; A61K 36/28; A61K 36/287; A61K 36/355; A61K 36/484; A61K 36/65; A61K 45/06

(57) ABSTRACT

A method and composition to extract of several processed herbal medicinal plants and an herbal tea composition for ingestion which contains the extract. The functional herbal extraction method and composition is used to relieve the symptoms of flu. This composition creates a synergy between different ingredients that works to suppress flu symptoms including headaches, cough, fever, sore throat and yellow phlegm. The composition comprises of Houttuynia cordata, Chrysanthemum, Paeoniae rubra, Coltsfoot flower, Gypsum, Pumice, Licoriei, and Honeysuckle. The effectiveness of the herbal tea is at least partially dependent on the desired means of preparation and administering the composition.

### FIELD OF THE INVENTION

The present invention relates generally to a flu relief herbal beverage method and composition. Further, the present invention relates to an herbal method and composition that balances the circulation of Qi and blood, balancing the lung cold and lung heat, thereby reducing phlegm and relieves cough, through a unique method of preparing a composition consisting of the following herbs in varying weight percentages including: Houttuynia cordata, Chrysanthemum, Paeoniae rubra, Coltsfoot flower, Gypsum, Pumice, Licoriei, and Honeysuckle.

### BACKGROUND OF THE INVENTION

Cough represents the primary motive for seeking medical care among outpatients. It serves as a potential indicator of both respiratory and non-respiratory ailments. Typically, a dry cough emerges during the latter stages of a cold, often attributed to conditions such as gastroesophageal reflux, post-nasal drip, or asthma. Traditional Chinese Medicine posits that lung vulnerability to external temperature changes triggers spasms in the alveolar mucosa, manifesting as bronchial spasms with thick or absent sputum, persisting for about a week. Presently, dry cough symptoms differ significantly, with many patients having a history of COVID-19 or influenza, and some experiencing effects from vaccinations. For these individuals, dry cough duration often extends beyond 3-4 weeks, occasionally persisting for months or even years, characterized as viral dry cough. Unlike traditional cough remedies, current treatments face challenges due to distinctions in causation and classification between past and contemporary dry coughs.

The Covid-19 virus can persist in a dormant state within the body, potentially leading to the reactivation of Covid symptoms in some cases. When these latent viruses are dormant and conditions such as decreased immunity or weather changes occur, they can become active again. Typically, patients experience a persistent dry cough with minimal sputum production during these periods. As the patient's immunity improves and their antiviral defenses strengthen, the virus may return to a dormant state, indicated when viral load falls below a certain threshold, such as 60 points. Consequently, dry cough symptoms may diminish or disappear. However, this cycle of recurring dry cough symptoms can be disruptive and distressing for the patient.

In Traditional Chinese Medicine (TCM), cough is understood as a symptom where the lungs respond to irritation, often attributed to a condition known as ‘lung counterflow qi.’ The lungs in TCM play a crucial role in dispersing and regulating Qi throughout the body. Their proper function relies on maintaining harmonious interactions with other organs, particularly the spleen and kidneys. Disruptions in this harmony can arise from external pathogens or internal imbalances. Phlegm accumulation in the lungs is thought to result from dampness obstructing the middle jiao, which stems from spleen dysfunction. When phlegm stagnates in the lungs, it impedes the descent of lung qi, leading to symptoms such as coughing, dyspnea, wheezing, chest tightness, and the production of copious sputum.

Traditional Chinese Medicine (TCM) has various methods for treating cough symptoms, but some of these methods may not align with modern biochemical understanding. For example, while TCM suggests that clam and snow pear soup can cure cough symptoms, it is not effective in cases of dry cough. This is because the sugar in fruits or desserts can accelerate the reproduction rate of dry cough viruses, thereby aggravating the symptoms.

Additionally, some individuals might use “Golden Throat” lozenges to alleviate throat pain. However, these lozenges contain sugar, which can also worsen dry cough by promoting virus replication. According to TCM theory, consuming sugar can damage the spleen, leading to dampness, which in turn generates phlegm, exacerbating cough symptoms. This explanation, although different from biochemical theories, arrives at a similar conclusion: sugar can worsen cough symptoms by promoting conditions that increase phlegm and virus activity.

### SUMMARY OF THE INVENTION

This invention generally relates to extracts of several processed herbal medicinal plants and an herbal tea composition for ingestion which contains the extract. The invention's composition is used to relieve the symptoms of cough and throat discomfort. This composition creates a synergy between different ingredients that works to suppress cough, white phlegm, and sore throat.

The invention is due, in part, to the unexpected finding that administration of the present invention's composition relieves cold cough and dry cough, diagnosable conditions in TCM. TCM diagnose cold, hot, dry, wet coughs by examining patients' pulse speed, tongue coating, color of the sputum, and color of the nasal mucus. The present invention's composition is at least used to relieve symptoms of cold cough and dry cough.

In a preferred embodiment of the present invention, the composition includes Houttuynia cordata, Chrysanthemum, Paeoniae rubra, Coltsfoot flower, Gypsum, Pumice, Licoriei, and Honeysuckle. The effectiveness of herbal tea is at least partially dependent on the desired means of preparation and administering the composition.

In one embodiment, the therapeutically effective amount comprises from between about 1 mg to about 14000 mg/day.

In a preferred embodiment of the present invention, the therapeutically effective amount comprises from between about 330 mg to 660 mm/day.

In a preferred embodiment of the present invention, the therapeutically effective amount is a dosage regiment of half of a bottle in the morning, and half of a bottle in the evening, wherein each bottle comprises about 330 mg of the composition.

In a preferred embodiment of the present invention, the therapeutically effective amount is a dosage regiment of one bottle in the morning, and one bottle in the evening, wherein each bottle comprises about 330 mg of the composition.

In one embodiment, the administration to the subject occurs between about 30 minutes to about 1 hour after consuming food.

In another embodiment, the composition is added with equal parts of water and heated over the stovetop or microwave, then administered to the subject.

In another preferred embodiment of the present invention, a method to brew an herbal beverage product has the following steps: the grinding together of all these aforementioned ingredients to obtain a dispersion of finely ground brewing particles, adding an aqueous solution to the ground particles, heating the aqueous solution from about room temperature to about boiling temperature, reduction of the aqueous liquid by simmering, filtering the aqueous liquid to obtain the first extract, adding an aqueous solution to the remaining solids of the first extract, heat the aqueous solution containing solids from the extract from about room temperature to about boiling temperature, reduction of the aqueous solution contain the first extract by simmering, filtering the aqueous solution containing solids from the first extract to obtain the second extract, combining both extracts with Caterpillar Fungus to create a third extract, reducing the third extract with a condensing machine.

In another preferred embodiment of the present invention, a method to brew an herbal beverage product has the following steps: the grinding together of all these aforementioned ingredients to obtain a dispersion of finely ground brewing particles, adding an aqueous solution to the ground particles, heating the aqueous solution from about room temperature to about boiling temperature, reduction of the aqueous liquid by simmering, filtering the aqueous liquid to obtain the extract.

In another preferred embodiment of the present invention, the composition is administered in at least one of the following ways: oral, topical, suppository, intravenous, intradermic, intramuscular, and intraperitoneal.

In another preferred embodiment of the present invention, the composition includes at least one member selected from the group consisting of: a tablet, an orally disintegrating tablet, a pill, a dripping pill, a lozenge, a capsule, a cream, a granule, a suppository, and an oral liquid.

In another preferred embodiment of the present invention, the composition consists of a plurality of herbal ingredients prepared through processing methods, extraction methods, and purification methods.

In another preferred embodiment of the present invention, the processing methods include at least one member selected from the group consisting of: washing, steaming, extracting, roasting, herb frying, salt frying, honey frying, wine frying, earth frying, vinegar frying, calcining drying, heating, and grinding.

In another preferred embodiment of the present invention, the extraction methods include at least one member selected from the group consisting of: soaking, heating, steaming, evaporation, compressing, and solvent extraction with at least one of the following: water, alcoholic solvents mixed with water, petroleum ether, hexane, diethyl amine, diethyl ether, cyclohexane, tert-butyl alcohol, isopropanol, Acetonitrile, acetone, ethanol, methyl isobutyl ketone, isobutyl alcohol, 1-propanol, methyl ethyl ketone, 2-butanol, isoamyl alcohol, 1-butanol, diethyl ketone, 1-octanol, p-xylene, m-xylene, toluene, dimethoxyethane, benzene, butyl acetate, 1-chlorobutane, tetrahydrofuran, ethyl acetate, o-xylene, hexamethylphosphorous triamide, 2-ethoxyethyl ether. N,N-dimethylacetamide, diethylene glycol dimethyl ether, N,N-dimethylformamide, 2-methoxyethanol, pyridine, propanoic acid, 2-methoxyethyl acetate, benzonitrile, 1-Methyl-2-pyrrolidinone, hexamethylphosphoramide, 1,4-dioxane, acetic acid, acetic anhydride, dimethyl sulfoxide, chlorobenzene, ethylene glycol, diethylene glycol, propylene carbonate, formic acid, 1,2-dichloroethane, glycerin, carbon disulfide, 1,2-dichlorobenzene, methylene chloride, nitromethane, 2,2,2-trifluoroethanol, chloroform, 1,1,2-trichlorotrifluoroethane, carbon tetrachloride, and tetrachloroethylene.

In another preferred embodiment of the present invention, the purification methods include at least one member selected from the group consisting of: column separation, solvent partition, evaporation, and solvent precipitation.

One objective of the present invention is to provide an herbal composition that suppresses and relieves cough symptoms.

Another objective is to provide an herbal composition that consists of easily available herbal ingredients.

Other systems, devices, methods, features, and advantages will be or become apparent to one with skill in the art upon examination of the following drawings and detailed description. It is intended that all such additional compositions, systems, methods, features, and advantages be included within this description, be within the scope of the present disclosure, and be protected by the accompanying claims and drawings.

### DETAILED DESCRIPTION OF THE INVENTION

Aspects of the invention are disclosed in the following description directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

The word “exemplary” is used herein to mean “serving as an example, instance, or illustration.” Any embodiment described herein as “exemplary” is not necessarily to be construed as preferred or advantageous over other embodiments. Likewise, the terms “embodiments of the invention.” “embodiments” or “invention” do not require that all embodiments of the invention include the discussed feature, advantage or mode of operation.

The following description directed to the medicinal properties of individual herbs and mixture of herbs is based, at least in part, on the teachings of Traditional Chinese Medicine (“TCM”). As such, the properties attributed to the individual herbs and mixtures of herbs set forth herein are done so in a non-limiting manner. Any evidence to the contrary based on other understandings or changes in understanding regarding TCM, or past and future scientific research will not operate to affect the operability of embodiments set forth and claimed herein, as will be readily recognized by one having ordinary skill in the art.

The invention is due, in part, to the unexpected finding that administration of the present invention's composition relieves cold cough, a diagnosable condition in TCM. If the patient's pulse speed is about 70 times a minute, combined with cough symptoms, the condition is diagnosed as a cold cough. Cold cough can also be diagnosed by looking at the color of the tongue coating. White tongue coating or no tongue coating indicates cold cough. The color of the sputum also indicates cold or hot cough. White phlegm or no phlegm indicates cold cough. Lastly, the color of the nasal mucus indicates cold cough or hot cough. If the phlegm is white or there is no phlegm, combined with cough symptoms, the condition is a cold cough. The present invention's composition is at least therapeutically effective in treating cold cough and dry cough.

For patients with dry cough, they are usually presented with tiredness, sleepiness, fatigue, weakness, lack of energy, and lack of concentration. This is also a typical symptom of the sequelae of COVID-19. For patients with dry cough, replenishing qi and blood, and restoring physical strength will improve immunity and reduces dry cough symptoms. Even when the symptoms of dry cough are cured, it will take at least three months for the patient to regain his previous physical strength. To completely eliminate dry cough, TCM should aim to regulate the spleen and stomach, strengthen the kidneys and replenish deficiency, moisten the lungs and replenish qi, strengthens the heart and replenish the yang, replenish Qi and blood. Especially for patients who are physically weak, accompanied by loss of appetite, or even eating less, they should focus on replenishing Qi. For patients who have developed chronic pneumonia or Cor pulmonale and rely on oxygen tanks, TCM should aim to increase the regeneration capacity of alveolar cells and get rid of oxygen cylinder supply as soon as possible.

The composition of the present invention consists of the following herbal ingredients mixed in varying weight percentages: Houttuynia cordata, Chrysanthemum, Paeoniae rubra, Coltsfoot flower, Gypsum, Pumice, Licoriei, and Honeysuckle. The herbal ingredients aforementioned combine to create a synergy to suppress cough symptoms, improve the circulation of Qi and blood, and balances the forces of Yin and Yang. The present disclosure provides a novel mixture of the ingredients, including unique processing, extraction, and purification methods for optimizing efficacy of the herbal ingredients. The effectiveness of the composition is at least partially dependent on the desired means of preparation and administering the composition.

The individual herbal ingredients are efficacious for nourishing the lung, regulating water in the physical body to reduce or eliminate phlegm. Those skilled in the art will recognize that TCM teaches that the lung is the primary organ that regulates the qi around the body. If the lung is obstructed, so is the qi that circulates in the body. The lung serves four functions of the qi, ascending, descending, inwards and outwards. The lung is an important organ to regulate both qi and blood.

According to the theories of TCM, the spleen produces phlegm and the lung stores phlegm. Cough, profusion of sputum and lung qi reversal occurs when the spleen does not properly dissolve dampness. The herbal composition works to improve balance between wet and dry to reduce and eliminate phlegm.

In a TCM clinical research setting, the composition of the present invention was administered to patients with cold cough and dry cough, about 95% to 98% of the patients experience the relief of cough symptoms within two to three weeks. In one study, an elementary school student was experiencing a cold and exhibited symptoms of cough and white phlegm. During TCM pulse diagnosis and tongue diagnosis, it was found that the lung meridian is weak and blocked, the student was experiencing dry cough, which worsened between 11 pm and 2 am. The student was administered with a total of 10 bottles of the composition, 2 bottles a day, and the symptoms were relieved.

In another case study, a TCM patient was complaining of swelling and pain in the right chest, caught a cold and developed fever, dizziness, tiredness, weakness, and tinnitus. The patient also had cold hands. The symptoms of cough showed dry cough, with white phlegm and the cough worsened at night. The pulse diagnosis showed that the lung and kidney meridians were weak and liver meridian was blocked. The patient was administered with a total of 7 bottles of the composition, 2 bottles a day, and the symptoms were relieved.

Houttuynia cordata (Yu Xing Cao) p. 216

Another herbal ingredient is Houttuynia cordata, known as Yuxingcao in mandarin. Yu Xing Cao is categorized as a heat-clearing, and toxin-eliminating herb. Yu Xing Cao is used in TCM to dispel heat and toxins, and drain abscesses and pus. Yu Xing Cao is sometimes used with Jie Geng (Radix platycodonis), Huang Qing (Radix scutellariae), and Lu Gen (Rhizoma phragmitis) to treat lung abscess. For coughing caused by Lung heat, Yu Xing Cao is used with Zhi Mu (Radix anemarrhenae), Zhe Bei Mu (Bulbus fritillariae thunbergii), and Sang Bai Pi (Cortex mori) to treat its symptoms. Yu Xing Cao, used in combination with Xing Ren (Semen armeniacae amarum), Shi Gao (Gypsum fibrosum) and Huang Qin (Radix scutellariae), are used to treat infections of the internal organs, including pneumonia, acute or chronic bronchitis, and enteritis.

Yu Xing Cao is chemically composed of essential oil 0.022 to 0.025% of decanoylacetaldehyde, methyl nonl ketone, laurinaldehyde, α-pinene, myrcene, limonene, linalool, citronellol, citral, 1,8-cine-oil, ocimene, carvacrol, thymol), and flavinoids (quercetin, isoquercitrin, quercitrin, reynoutrin, hyperin, rutin, cordarine). Houttuynium is the synthetic sodium derivative of decanoylacetaldehyde.

Yu Xing Cao has a broad spectrum of action to inhibit Staphylococcus aureus, β-hemolytic streptococcus, Diplococcus pneumoniae, Neisseria catarrhalis, Corynebacterium diphtheriae, Bacillus proteus, Bacillus dysenteriae, leptospira, dermatophytes and some influenza viruses. Administration of Yu Xing Cao in rabbits is associated with an increase in phagocytic activities by the white blood cells, and an increase of serum properdin.

In one study, 25 of 28 patients with pneumonia showed complete recovery following an herbal treatment containing 30 grams of Yu Xing Cao and 15 grams of Jie Geng (Radix platycodonis). The herbs were cooked in water to yield 200 ml of decoction. Patients were given 30 ml per dose, three to four times daily. Those with a profuse amount of thick sputum were also treated with inhalation of a 5% Yu Xing Cao solution.

In another study, 190 patients with chronic bronchitis were divided into two groups and treated with houttuynium. In the first group, the study reported a 78% rate of effectiveness with intramuscular injection. In the second group, the study reported a 76.7% from oral ingestion of houttuynium.

### Chrysanthemum (Juhua) p. 75

Another herbal ingredient is Chrysanthemum, known as Ju Hua in mandarin. Ju Hua is categorized as a Wind-Heat dispelling, herb. Ju Hua is commonly used to dispel wind-heat and treat early-stage febrile disorders and wind-heat syndromes characterized by headache, fever, red and painful eyes, dizziness, dry mouth and throat, and a yellow tongue coat. Ju Hua clears the liver and benefits the eyes, calms Liver Yang, and clears heat and eliminates toxins. To treat infertility and wind-heat syndrome, Ju Hua is used with Sang Ye (Folium mori), Bo He (Herba menthae), Lu Gen (Rhizoma phragmitis), and Lian Qiao (Fuctus forsythiae).

Ju Hua is one of the most commonly used herbs in treatment of eye disorders. It treats eye disorders caused by wind-heat attacking the Liver channel, or Liver fire rising, with symptoms such as redness, swelling and pain in the eyes, superficial visual obstruction, and increased production of tears on exposure to wind. It treats blurred vision and dizziness from Liver and Kidney yin deficiencies. It is, however, contraindicated if the diagnosis is yang deficiency, or deficiency and cold.

Ju Hua is chemically composed of Essential oils 0.2-0.85% (borneol, bornyl acetate, chrysanthenone, camphor), flavone (luteolin, apigenin, acacetin), chrysanthemin A, B; chlorochrymorin, chrysandiol. Pharmacologically, Ju Hua is vasodilative. Ju Hua has been shown to dilate blood vessels, increase blood perfusion to the coronary arteries, lowers blood pressure, and decrease body temperature. Ju Hua is also antibiotic—it has been shown to inhibit Staphylococcus aureus, β-hemolytic streptococcus, and Shigella sonnei. It also has inhibitory effect against leptospira at high doses.

According to one study, 164 patients with coronary artery disease were treated with a preparation of Ju Hua (containing the equivalent of 50 grams of dried herb) for 2 months per treatment course, with a total treatment duration ranging between 1 and 2 courses. The study reported improvement based on symptom evaluation in 86.5% of patients, and improvement based on EKG evaluation in 45.3% of patients.

Paeoniae rubra (Chishao) p. 162

Another herbal ingredient is Paeoniae rubra, known as Chi Shao in mandarin. Chi Shao is categorized as a heat-clearing, blood-cooling, pain-relieving herb. Chi Shao dispels blood stasis, reduces swelling from sores and abscesses, relieves eye pain. Chi Shao treats various bleeding disorders caused by febrile disorders or heat attacking the blood. Chi Shao is often used to treat amenorrhea and dysmenorrhea. Blood stagnation in uterus leads to amenorrhea or dysmenorrhea with blood clots. The blood is often dark in color, and pain is alleviated after clots have been dispelled. To treat this condition, Chi Shao is added with Dang Gui (Radicis angelicae sinensis) and Chuan Xiong (Rhizoma ligustici).

Chi Shao is chemically composed of paeoniflorin, benzoylpaeoniflorin, oxypaeoniflorin, lactiflorin, daucosterol, d-catechin. The pharmacological effects of Chi Shao are antiplatelet, hematological, cardiovascular, antispasmodic, antibiotic, antiseizure and antipyretic effects. In one study, 125 patients with coronary artery disease were treated with 40 grams of Chi Shao decoction three times daily, with satisfactory results. In another study, intravenous infusion of Chi Shao injectables with D5W or D10W once daily was 92.01% effective for prevention of thrombus formation in 263 patients. According to one report, 10 grams of Chi Shao given three times daily was effective in treating 30 patients with cor pulmonale.

### Coltsfoot Flower (Kuan Dong Hua)

Another herbal ingredient is Coltsfoot flower, known as Kuan Dong Hua in mandarin. Kuan Dong Hua is categorized as a coughing/wheezing-releasing-herb. Kuan Dong Hua is used in TCM to dissolve phlegm, stops cough due to lung cold, lung heat, or lung yin deficiency. It is most effective for treating cough due to cold attacking the lung, or chronic cough with blood-streaked sputum. When combined with appropriate herbs, Kuan Dong Hua treats upper respiratory infection, influenza, and a wide range of coughing disorders, including cold or hot, exterior or interior, and excess or deficient conditions. In a clinical study, extract of Kuan Dong Hua (equivalent to 6 grams of herb) given three times daily provided marked relief in treating 36 asthma patients. The preparation, however, was ineffective for treating acute and severe onset of an asthma attack.

### Gypsum (Shi Gao)

Another herbal ingredient is Gypsum, known as Shi Gao in mandarin. Shi Gao is categorized as heat-clearing and fire-purging herbs, used in TCM to clear heat, sedates fire, relieves irritability and quenches thirst, clears lung heat, clears stomach heat and channel, clears heat and promotes healing from sores, burns, and eczema. Gypsum has low solubility than other herbs so it is common to use in larger dosage. However, if gypsum is administered orally, the dosage is lowered since solubility is no longer applicable.

Shi Gao reduces heat due to stroke, dyspnea in the heart, and asthma, wheezing and sore throat. The pharmacological effects of Shi Gao are antipyretic, immunostimulant, and shown to facilitate the passage of bile, shorten coagulation time, promote diuresis, and reduce spontaneous intake of water in laboratory mice. According to one report, 200 patients with high fever were treated with an herbal decoction (120 grams of Shi Gao, 3 grams of Ma Huang, and 3 grams of Guizhi. Of 200 patients, 181 showed a reduction in body temperature.

Pumex preparata (Haifushi)

Another herbal ingredient is Pumex preparata, known as Haifushi in mandarin. Haifushi is categorized as phlegm-resolving herb, used in TCM for clearing the lung, dissolving phlegm, softens hardness and dissipate nodules, regulates water circulation to treat dysuria.

Radix glycyrrhizae (Gan Cao)

Another herbal ingredient is Radix flycyrrhizae, known as Gan Cao in mandarin. Gan Cao is categorized as spleen-tonifying, lung moistening, and a pain-relieving herb, used in TCM to benefit Qi, to treat spleen and stomach deficiencies, stops cough and pain. Gan Cao is neutral in property and mild in action. It treats coughing and wheezing of various etiologies, including cold or heat, and deficiency or excess, with or without phlegm. Gan Cao is also used to treat epigastric, abdominal, musculoskeletal muscle pain and cramps. Gan Cao's pharmacological effects include mineralocorticoid, glucocorticoid, anti-inflammatory, antiarrhythmic, immunologic, gastrointestinal, antispasmodic, antitoxin, hepatoprotective, antitussive and expectorant, analgesic, antibiotic, and antihyperlipidemic.

In one study, 8 out of 9 patients with declining pituitary function were treated successfully by 2 to 3 months of taking 15 to 30 grams of Gan Cao and 6 grams of Radix ginseng, and for severe symptoms, an additional 10 grams of Radix aconiti Lateralis Preparata. For those with elevated blood pressure, the dosage of Gan Cao was reduced by 50% and 10 grams of Poria, 10 grams of Cortex eucommiae, 10 grams of Fuctus schisandrae Chinensis were added.

In another study, 55 patients with pulmonary tuberculosis were treated with an herbal decoction three times daily for 30 to 90 days. There was significant improvement in 23 patients and some improvement in 32 patients.

### Honeysuckle (Jinyinhua)

Another herbal ingredient is honeysuckle, also known as Flos lonicerae, also known as Jin Yin Hua in mandarin. Jin Yin Hua is categorized as a heat-clearing herb used in TCM to clear heat, eliminate toxins, eliminate sores and abscesses, diarrhea or dysentery. Combined with other appropriate herbs, Jin Yin Hua can be used for treatment of many heat conditions. Clinical manifestations of heat include wind-heat and early-stage febrile disorders with sore throat, fever perspiration and thirst; heat in the qi (energy) level showing as high fever, thirst, perspiration and forceful pulse; summer-heat with fever, heaviness sensations and heat stroke; and heat in the ying (nutritive) and xue (blood) levels with irritability, insomnia, bleeding, purpura or macules on the skin, with a scarlet red tongue. Though Jin Yin Hua is useful against all types of heat, it is most commonly used and most effective against the initial stages of febrile disorders, in which heat is in the upper jiao, at the wei (defense) and qi (energy) levels.

Jin Yin Hua is commonly used to clear heat and eliminate toxins from the interior and the exterior. The combination of heat and toxins in the interior is characterized by sore throat, swelling, intestinal abscesses and lung abscesses at various stages of development. Toxic heat in the exterior is characterized by all kinds of dermatological sores, lesions, ulcerations, warts and furuncles. Jin Yin Hua is also used for syphilis. Some exemplary formulas, including Wu Wei Xiao Du Yin (Five-Ingredient Decoction to Eliminate Toxins) and Zhen Ren Huo Ming Yin (True Man Decoction to Revitalize Life), uses Jin Yin Hua in combination including Ju Hua (Chrysanthemum) and Pu Gong Ying (Herba taraxaci) and Zi Hua Di Ding (Herba violae) to treat sores, lesions, carbuncles, and ulcerations of the skin.

Heat and toxins attacking the intestines often leads to diarrhea or dysentery, and rectal tenesmus sometimes with the presence of mucus and blood. For diarrhea with mucus but no blood, Jin Yin Hua with sugar is used to treat the symptoms. For Diarrhea with blood, Jin Yin Hua is used with brown sugar to treat the symptoms. For diarrhea or dysentery with mucus due to damp-heat and toxins, Jin Yin Hua is used in combination with Huang Lian (Rhizoma coptidis), Huang Qin (Radix scutellariae), Ge Gen (Radix puerariae), Mu Xiang (Radix aucklandiae), Ma Chi Xian (Herba portulacae) and Bai Tou Weng (Radix pulsatillae) to treat its symptoms.

Jin Yin Hua is chemically composed of Lonicein, loniceraflavone, loganin, tannin, chlorogenic acid, isochlorogenic acid, aromadendrene, linalool, geraniol, octanal, α-pinene, β-pinen, myrcene, 1,80-cineole, β-terpinene, hexenol, α-terpineol, neral, linalyl acetate, geranial, citronellol, terpinyl acetate, eugenol, β-eudesmol, geranyl acetate, α-copene, patchoulene, α-caryophyllene, β-caryophyllene, iso-bornyl acetate, farnesol, nerolidol.

Jin Yin Hua has demonstrated a broad spectrum of inhibitory actions against Staphylococcus aureus, β-hemolytic streptococcus, E. coli, Bacillus dysenteriae, Vibro cholerae, Salmonella typhi, Diplococcus pneumoniae, Diplococcus meningitidis, Pseudomonas aeruginosa, and Mycobacterium tuberculosis. Chlorogenic acid and isochlorogenic acid have the strongest antibiotic effects. The fresh herb (just soaked in water) has a stronger antibiotic action than herbal decoction. Jin Yin Hua is antibiotic, anti-inflammatory, antipyretic, CNS stimulant, antihyperlipidemic, and it has a gastrointestinal effect.

The extract of each of the above identified herbs is prepared and decocted by traditional procedures known in the art which includes boiling, filtering, and forming concentrates.

In a preferred embodiment of the present invention, a method to brew an herbal beverage product has the following steps: the grinding together of all these aforementioned ingredients to obtain a dispersion of finely ground brewing particles, adding an aqueous solution to the ground particles, heating the aqueous solution from about room temperature to about boiling temperature, reduction of the aqueous liquid by simmering, filtering the aqueous liquid to obtain the first extract, adding an aqueous solution to the remaining solids of the first extract, heat the aqueous solution containing solids from the extract from about room temperature to about boiling temperature, reduction of the aqueous solution to obtain the extract.

To form the composition of the present invention in one embodiment, the extracted concentrate of each herb is mixed according to the following measurements: Houttuynia cordata, Chrysanthemum, Paeoniae rubra, Coltsfoot flower, Gypsum, Pumice, Licoriei, and Honeysuckle, about 10 to 20 percent by dry weight,

The composition of the present invention is for ingestion in the form of a tea, liquid beverage, tablet, capsule, powder, or soup. Conventionally known methods known in the art are used to prepare the composition in the different forms. The form in which the composition is prepared does not significantly affect its efficacy. The ideal form of the composition is an herbal tea. For mild symptoms, ½ of the preferred quantity should be consumed in the morning, and ½ of the preferred quantity should be consumed in the evening. The ideal form of the composition, in an herbal tea, is to mix with 1 part water and 1 part composition, heated over the countertop or microwave, and consumed after a meal. The composition should be taken daily on a regular basis to obtain maximum benefits, until the cough symptom alleviates.

While a preferred embodiment of the invention has been described and illustrated for purposes of clarity and example, it should be understood that many changes, substitutions and modifications to the described embodiment will be apparent to those having skill in the art in light of the foregoing disclosure without departing from the scope and spirit of the present invention which is defined by the claim which will follow.

What is claimed is:

1. A method for preparing an herbal cough relief beverage composition comprising the steps of:
Adding together herbal ingredients comprising Houttuynia cordata, Chrysanthemum, Paeoniae rubra, Coltsfoot flower, Gypsum, Pumice, Licoriei, and Honeysuckle. Add an aqueous solution to the ingredients. Heat the aqueous solution from about room temperature to about boiling temperature. Reduction of the aqueous liquid by simmering. Filtering the aqueous liquid to obtain the extract.

2. The method of claim 1, wherein the ingredients further comprise at least one additive selected from the group consisting of caloric sweetener, non-caloric sweetener, flavoring, coloring, preservative, caffeine, guarana, vitamin, dietary mineral, amino acid, protein, taurine, omega-3 fatty acid, essential fatty acid, polyunsaturated fat, monounsaturated fat, and medium chain triglyceride.

3. The method of claim 1, wherein the brewing ingredient comprises a summation of one or more members selected from the group consisting of tea leaves, herb, spice, dried fruit, dried vegetable, and cacao.

4. The method of claim 1, wherein the brewing ingredient comprises at least one polysaccharide selected from the group consisting of inulin, chicory root extract, acacia gum, ester gum, gellan gum, guar gum, locust bean gum, tara gum, xanthan gum, agar, alginin, arrowroot, carrageenan, dextrin, maltodextrin, methyl cellulose, pectin, polydextrose, stevia, stevia extract, tapioca, oligosaccharide, starch, thickener, and vegetable gum.

5. The method of claim 1, wherein the brewing ingredient comprises a summation of one or more members selected from the group consisting of non-dried fruit and non-dried vegetable.

6. A composition for provisioning an herbal tea for suppression of cough symptoms, comprising a mixture of plant extracts, where in the mixture of plant extracts consist of at least one Houttuynia cordata, at least one Chrysanthemum, at least one Paeoniae rubra, at least one Coltsfoot flower, at least one Gypsum, at least one Pumice, at least one Licoriei, and at least one Honeysuckle; and
Adding the herbal mixture to an aqueous liquid.

7. The composition of claim 6, wherein the composition is administered in at least one of the following ways: oral, topical, suppository, intravenous, intradermic, intramuscular, and intraperitoneal.

8. The composition of claim 6, wherein the composition includes at least one member selected from the group comprising of: a liquid beverage, an aqueous beverage, a tablet, an orally disintegrating tablet, a pill, a dripping pill, a lozenge, a capsule, a cream, a granule, and a suppository.

9. The composition of claim 6, wherein the composition consists of a plurality of herbal ingredients prepared through processing methods, extraction methods, and purification methods.

10. The composition of claim 6, wherein the processing methods include at least one member selected from the group consisting of: purification, grinding, pulverizing, soaking, heating, evaporation, compressing, washing, steaming, extracting, roasting, herb frying, salt frying, honey frying, wine frying, earth frying, vinegar frying, calcining and drying.
